(19) United States
(12) Patent Application Publication
(10) Pub. No.: US 2024/0122885 A1
(43) Pub. Date: Apr. 18, 2024

(54) Endometriosis-Related Methods and Compositions

(71) Applicant: The Regents of the University of California, Oakland, CA (US)

(72) Inventors: Marina Sirota, San Francisco, CA (US); Linda Giudice, San Francisco, CA (US); Arohee Bhoja, San Francisco, CA (US); Daniel Bunis, San Francisco, CA (US)

(21) Appl. No.: 18/276,834

(22) PCT Filed: Feb. 11, 2022
(86) PCT No.: PCT/US2022/016175

§ 371 Date: Aug. 10, 2023

Related U.S. Application Data
(60) Provisional application No. 63/149,022, filed on Feb. 12, 2021.

(51) Int. Cl. A61K 31/192; A61P 15/00

(52) U.S. Cl. CPC A61K 31/192; A61P 15/00

(57) ABSTRACT

Provided are methods of modulating gene expression levels in an individual identified as having endometriosis. The methods comprise administering to the individual identified as having endometriosis a drug described herein in an amount effective to modulate gene expression levels in the individual. Also provided are pharmaceutical compositions. The compositions comprise a drug described herein in an amount effective to modulate gene expression levels in an individual, wherein the pharmaceutical composition is adapted for intrauterine or intravaginal administration of the drug to the individual. Kits that find use in practicing the methods of the present disclosure are also provided. In some embodiments, the kits comprise a pharmaceutical composition comprising a drug described herein in an amount effective to modulate gene expression levels in the individual, and instructions for administering the pharmaceutical composition to an individual identified as having endometriosis.

### CROSS-REFERENCE TO RELATED APPLICATIONS

This application claims the benefit of U.S. Provisional Patent Application No. 63/149,022, filed Feb. 12, 2021, which application is incorporated herein by reference in its entirety.

### STATEMENT OF GOVERNMENT SUPPORT

This invention was made with Government support under contract 5P50HD055764-13 awarded by the National Institutes of Health. The Government has certain rights in the invention.

### INTRODUCTION

Endometriosis is a reproductive disease characterized by growth of endometrial tissue outside its normal location, which leads to symptoms of pelvic scarring, pain, and infertility. Although it is extremely common, affecting over 200 million people worldwide, current treatments mostly focus on symptom management and are not always effective [1]. Traditional drug discovery methods tend to be expensive and time consuming; it can take up to 15 years and $1 billion to bring a new drug to market [2]. Additionally, many of these novel drugs fail in later stages of testing, resulting in a significant loss of money and time.

### SUMMARY

Provided are methods of modulating gene expression levels in an individual identified as having endometriosis. The methods comprise administering to the individual identified as having endometriosis a drug described herein in an amount effective to modulate gene expression levels in the individual. Also provided are pharmaceutical compositions. The compositions comprise a drug described herein in an amount effective to modulate gene expression levels in an individual, wherein the pharmaceutical composition is adapted for intrauterine or intravaginal administration of the drug to the individual. Kits that find use in practicing the methods of the present disclosure are also provided. In some embodiments, the kits comprise a pharmaceutical composition comprising a drug described herein in an amount effective to modulate gene expression levels in the individual, and instructions for administering the pharmaceutical composition to an individual identified as having endometriosis.

### BRIEF DESCRIPTION OF THE FIGURES

FIG. 1: A flow diagram providing an overview of the pipeline employed to identify novel therapeutic candidates for endometriosis.

FIG. 2: UpSet plot showing overlap in drug hits across signatures.

FIG. 3: Heatmap showing reversal scores for top 20 drugs across all signatures.

FIG. 4: Bar plot showing distributions of drug classes.

FIG. 5: Drug target network for top 20 combined drug hits.

FIG. 6: Median escape response (%) with interquartile range for each delivered volume (0.01, 0.15, 0.30, 0.40, 0.55, 0.70, 0.80, and 0.90 mL) during the baseline, post-endo surgery, and post-treatment periods with A) Fenoprofen treatment, B) Ibuprofen treatment (positive control), C) No endo surgery and no treatment (negative control), D) No treatment (negative control). Volume is the volume of water delivered to the rat's vaginal canal (via balloon). % Escape response is the number of times a rat responded to a specific volume. All “volumes” (8 volumes: 7 volumes and 1 sham) are delivered randomly 3 times each in a 1 hour testing session or “run”.

### DETAILED DESCRIPTION

Before the methods, compositions and kits of the present disclosure are described in greater detail, it is to be understood that the methods, compositions and kits are not limited to particular embodiments described, as such may, of course, vary. It is also to be understood that the terminology used herein is for the purpose of describing particular embodiments only, and is not intended to be limiting, since the scope of the methods, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, compositions and kits. The upper and lower limits of these smaller ranges may independently be included in the smaller ranges and are also encompassed within the methods, compositions and kits, subject to any specifically excluded limit in the stated range. Where the stated range includes one or both of the limits, ranges excluding either or both of those included limits are also included in the methods, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, compositions and kits belong. Although any methods, compositions and kits similar or equivalent to those described herein can also be used in the practice or testing of the methods, compositions and kits, representative illustrative methods, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, compositions and kits, which are, for clarity, described in the context of separate embodiments, may also be provided in combination in a single embodiment. Conversely, various features of the methods, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

The present disclosure provides methods of modulating gene expression levels in an individual identified as having endometriosis. In certain embodiments, the methods comprise administering to the individual identified as having endometriosis a drug disclosed herein (e.g., a drug from Table 2) in an amount effective to modulate gene expression levels in the individual. As demonstrated in the Experimental section herein, the inventors elucidated gene expression signatures for patients with stage ½ and ¾ endometriosis at the early secretory (ESE), mid-secretory (MSE), and proliferative (PE) phases in the cycle, and further identified the drugs provided in Tables 2, 3 and 4 as those known to have opposite transcriptional effects with respect to the endometriosis gene expression signatures. That is, the drugs in Tables 2, 3 and 4 are reasonably expected to normalize (partially or completely) gene expression patterns associated with endometriosis, and therefore find use in modulating gene expression levels in individuals identified as having endometriosis. Moreover, as proof of concept and also demonstrated in the Experimental section herein, a drug identified by the inventors as having an opposite transcriptional effect with respect to the endometriosis gene expression signature was determined to be efficacious in an established animal model of endometriosis. Accordingly, in certain embodiments, the methods find use in treating endometriosis, e.g., resulting in at least an amelioration of one or more symptoms of endometriosis in the individual. Details regarding the methods of the present disclosure will now be provided.

As summarized above, a drug disclosed herein (e.g., a drug in Table 2) is administered to an individual identified as having endometriosis. By “an individual identified as having endometriosis” is meant it is known, prior to administering the drug, that the individual has endometriosis, and the drug is administered to the individual on the basis that the individual has endometriosis. The types of individuals may vary and generally include human females of reproductive age. For example, the individual may be a human female of age 20-60, e.g., 25-55, such as a human female 30-40 years of age.

According to some embodiments, the methods further comprise identifying the individual as having endometriosis. Methods of identifying (diagnosing) endometriosis are known and may be based upon the individual's symptoms (e.g., dysmenorrhea, infertility, chronic pelvic pain, abnormal uterine bleeding or spotting, pain during intercourse, painful urination during menstrual periods, painful bowel movements during menstrual periods, and/or the like), manual pelvic examination (e.g., palpation for a fixed, retroverted uterus, adnexal and uterine tenderness, pelvic masses or nodularity along the uterosacral ligaments), rectovaginal examination (e.g., to identify uterosacral, cul-de-sac or septal nodules), pelvic ultrasonography (e.g., transvaginal ultrasound to identify endometriomas), magnetic resonance imaging (MRI), laparoscopy (e.g., by insertion of a laparoscope through an insertion near the naval providing information about the location, extent and size of the endometrial implants), known biomarkers for endometriosis, and any combination thereof.

According to some embodiments, the individual has been identified as having stage ½ endometriosis. Stage 1 (or minimal) is typically characterized by a few small implants or small wounds or lesions. They may be found on the organs or the tissue lining the pelvis or abdomen, with little to no scar tissue. Stage 2 (or mild) is typically characterized by more implants than in stage 1, which may be deeper in the tissue, and there also may be some scar tissue. In certain embodiments, when the individual has been identified as having stage ½ endometriosis, the method comprises administering to the individual an effective amount of a drug from Table 3.

In certain embodiments, the individual has been identified as having stage ¾ endometriosis. Stage 3 (or moderate) is typically characterized by many deep implants, possibly including small cysts on one or both ovaries, and thick bands of scar tissue called adhesions. Stage 4 (or severe) is typically characterized by many deep implants and thick adhesions, as well as large cysts on one or both ovaries. According to some embodiments, when the individual has been identified as having stage ¾ endometriosis, the method comprises administering to the individual an effective amount of a drug from Table 4.

The drug (e.g., a drug from Table 2, 3 or 4) is administered in an amount effective to modulate gene expression levels in the individual. By “effective amount” or “therapeutically effective amount” is meant a dosage sufficient to produce a desired result, e.g., modulate gene expression levels in the individual (e.g., an amount effective to normalize (partially or completely) the individual's gene expression patterns associated with endometriosis), an amount sufficient to effect beneficial or desired therapeutic (including preventative) results, such as a reduction in a symptom of endometriosis. For example, the drug may be administered in an amount effective to modulate gene expression resulting in at least an amelioration of one or more symptoms of endometriosis in the individual. Non-limiting examples of symptoms which may be ameliorated according to the methods of the present disclosure include dysmenorrhea, infertility, chronic pelvic pain, abnormal uterine bleeding or spotting, pain during intercourse, painful urination during menstrual periods, painful bowel movements during menstrual periods, and any combination thereof. The drugs provided in Tables 2, 3 and 4 are known, as is information regarding their pharmacokinetics and the like which a physician may use to determine a suitable dosage and/or dosage regimen to modulate (e.g., normalize partially or completely) gene expression levels associated with endometriosis as desired in the individual.

According to some embodiments, the methods are effective in treating the endometriosis of the individual. By “treat” or “treatment” is meant at least an amelioration of the symptoms associated with the endometriosis, where amelioration is used in a broad sense to refer to at least a reduction in the magnitude of a parameter, e.g., symptom, associated with the endometriosis being treated. As such, treatment also includes situations where the endometriosis, or at least symptoms associated therewith, are completely inhibited, e.g., prevented from happening, or stopped, e.g., terminated, such that the individual no longer suffers from the endometriosis, or at least the symptoms that characterize the endometriosis.

Dosing may be dependent on severity and responsiveness of the disease state to be treated. Optimal dosing schedules can be calculated from measurements of drug accumulation in the body of the individual. The administering physician can determine optimum dosages, dosing methodologies and repetition rates. Optimum dosages may vary depending on the relative potency of individual therapeutic agents, and can generally be estimated based on EC₅₀s found to be effective in in vitro and in vivo animal models, etc. In general, dosage is from about 0.01 μg to about 100 g per kg of body weight, and may be given once or more daily, weekly, monthly or yearly. In certain aspects, the dosage is from about 1 μg/kg to 100 mg/kg or more, depending on the factors mentioned above. The treating physician can estimate repetition rates for dosing based on measured residence times and concentrations of the therapeutic agent in bodily fluids or tissues. Following successful treatment, it may be desirable to have the subject undergo maintenance therapy to prevent the recurrence of the disease state, where the therapeutic agent is administered in maintenance doses, ranging from about 0.01 μg to about 100 g per kg of body weight, once or more daily, to once every several months, once every six months, once every year, or at any other suitable frequency.

The therapeutic methods of the present disclosure may include administering a single type of therapeutic agent to the individual, or may include administering two or more types of therapeutic agents to the individual separately or by administration of a cocktail of different therapeutic agents. For example, in certain embodiments, two or more drugs independently selected from Table 2, 3 or 4 may be administered to the individual, e.g., two or more, three or more, four or more, or five or more drugs from Table 2, 3 or 4. According to some embodiments, one or more (e.g., two or more) drugs from Table 2, 3 or 4 are administered to the individual, in combination with an existing treatment for endometriosis. Such existing treatments include, but are not limited to, administration of an effective amount of a GnRH agonist (e.g., Leuprolide acetate (Lupron), Goserelin acetate (Zoladex), Nafarelin acetate (Synarel), and/or the like), a progestin (e.g., depot medroxyprogesterone acetate (Depo-Provera), norethindrone acetate (Aygestin), and/or the like), Danazol, surgery, or any combination thereof.

The one or more drugs may be administered to an individual using any available method and route suitable for drug delivery, including in vivo and ex vivo methods, as well as systemic and localized routes of administration. Conventional and pharmaceutically acceptable routes of administration include oral and parenteral routes of administration. Parenteral routes of administration of interest include, but are not limited to, injection (e.g., intravenous, intra-arterial, local, subcutaneous, or intramuscular injection), intrauterine, intravaginal, intranasal, intra-tracheal, intradermal, topical application, ocular, nasal, and other parenteral routes of administration. Routes of administration may be combined, if desired, or adjusted depending upon the drug and/or the desired effect. The drug may be administered in a single dose or in multiple doses. In some embodiments, the drug is administered intravenously. In some embodiments, the drug or pharmaceutical composition is administered by injection, e.g., for systemic delivery (e.g., intravenous infusion) or to a local site.

According to some embodiments, the drug is administered to the individual by intrauterine administration. By way of example, intrauterine administration may comprise placement of an intrauterine device (IUD) comprising one or more drugs from Tables 2, 3, or 4 (e.g., flumetasone, primaquine, flunisolide, zuclopenthixol, irinotecan, scopolamine, promazine, or any combination thereof) releasable therefrom in the uterus of the individual. Approaches and considerations for intrauterine drug delivery (including the use of IUDs) are known and described, e.g., in ESHRE Capri Workshop Group (2008) Hum Reprod Update (“Intrauterine devices and intrauterine systems”) 14(3):197-208; Davis (2011) Cuff Opin Pediatr. 23(5):557-65; Wu & Pickle (2014) Contraception 89(6):495-503; Mishell (1998) Contraception 58(3 Suppl):45S-53S; Grimes et al. (2010) Cochrane Database Syst Rev. (6):CD001777; the disclosures of which are incorporated herein by reference in their entireties for all purposes.

In certain embodiments, the drug is administered to the individual by intravaginal administration. Non-limiting examples of intravaginal administration include administering (e.g., by placement inside the vagina) a vaginal suppository comprising the drug releasable therefrom or a vaginal ring comprising the drug releasable therefrom to the individual. Approaches and considerations for intravaginal drug delivery are known and described, e.g., in Woolfson & Gallagher (2000) Crit Rev Ther Drug Carrier Syst. 17(5):509-55; Friend (2011) Drug Deliv Transl Res. 1(3):185-93; Hussain & Ahsan (2005) J Control Release. 103(2):301-13; Mohideen et al. (2017) Biomaterials. 144:144-154; das Neves & Bahia (2006) Int J Pharm. 318(1-2):1-14; Alexander et al. (2004) Fertil Steril. 82(1):1-12; and de Araujo Pereira & Bruschi (2012) Drug Dev Ind Pharm. 38(6):643-52; the disclosures of which are incorporated herein by reference in their entireties for all purposes. For example, known approaches and formulations that may be employed for intravaginal delivery of one or more drugs from Tables 2, 3, or 4 (e.g., flumetasone, primaquine, flunisolide, zuclopenthixol, irinotecan, scopolamine, promazine, or any combination thereof) include hydrogels, vaginal tablets, pessaries/suppositories, particulate systems, and intravaginal rings.

In certain embodiments, provided are methods of modulating gene expression levels in an individual identified as having endometriosis, the methods comprising administering to the individual identified as having endometriosis flumetasone in an amount effective to modulate gene expression levels in the individual. The flumetasone may be administered in an amount effective to normalize (partially or completely) the gene expression pattern associated with endometriosis as desired in the individual. According to some embodiments, the flumetasone is administered in an amount effective to treat the endometriosis of the individual, e.g., by resulting in at least an amelioration of one or more symptoms of endometriosis in the individual.

According to some embodiments, provided are methods of modulating gene expression levels in an individual identified as having endometriosis, the methods comprising administering to the individual identified as having endometriosis primaquine in an amount effective to modulate gene expression levels in the individual. The primaquine may be administered in an amount effective to normalize (partially or completely) the gene expression pattern associated with endometriosis as desired in the individual. According to some embodiments, the primaquine is administered in an amount effective to treat the endometriosis of the individual, e.g., by resulting in at least an amelioration of one or more symptoms of endometriosis in the individual.

In certain embodiments, provided are methods of modulating gene expression levels in an individual identified as having endometriosis, the methods comprising administering to the individual identified as having endometriosis flunisolide in an amount effective to modulate gene expression levels in the individual. The flunisolide may be administered in an amount effective to normalize (partially or completely) the gene expression pattern associated with endometriosis as desired in the individual. According to some embodiments, the flunisolide is administered in an amount effective to treat the endometriosis of the individual, e.g., by resulting in at least an amelioration of one or more symptoms of endometriosis in the individual.

According to some embodiments, provided are methods of modulating gene expression levels in an individual identified as having endometriosis, the methods comprising administering to the individual identified as having endometriosis zuclopenthixol in an amount effective to modulate gene expression levels in the individual. The zuclopenthixol may be administered in an amount effective to normalize (partially or completely) the gene expression pattern associated with endometriosis as desired in the individual. According to some embodiments, the zuclopenthixol is administered in an amount effective to treat the endometriosis of the individual, e.g., by resulting in at least an amelioration of one or more symptoms of endometriosis in the individual.

In certain embodiments, provided are methods of modulating gene expression levels in an individual identified as having endometriosis, the methods comprising administering to the individual identified as having endometriosis irinotecan in an amount effective to modulate gene expression levels in the individual. The irinotecan may be administered in an amount effective to normalize (partially or completely) the gene expression pattern associated with endometriosis as desired in the individual. According to some embodiments, the irinotecan is administered in an amount effective to treat the endometriosis of the individual, e.g., by resulting in at least an amelioration of one or more symptoms of endometriosis in the individual.

According to some embodiments, provided are methods of modulating gene expression levels in an individual identified as having endometriosis (e.g., identified as having stage ½ endometriosis, or identified as having stage ¾ endometriosis), the methods comprising administering to the individual identified as having endometriosis scopolamine in an amount effective to modulate gene expression levels in the individual. The scopolamine may be administered in an amount effective to normalize (partially or completely) the gene expression pattern associated with endometriosis as desired in the individual. According to some embodiments, the scopolamine is administered in an amount effective to treat the endometriosis of the individual, e.g., by resulting in at least an amelioration of one or more symptoms of endometriosis in the individual.

In certain embodiments, provided are methods of modulating gene expression levels in an individual identified as having endometriosis (e.g., identified as having stage ½ endometriosis), the methods comprising administering to the individual identified as having endometriosis promazine in an amount effective to modulate gene expression levels in the individual. The promazine may be administered in an amount effective to normalize (partially or completely) the gene expression pattern associated with endometriosis as desired in the individual. According to some embodiments, the promazine is administered in an amount effective to treat the endometriosis of the individual, e.g., by resulting in at least an amelioration of one or more symptoms of endometriosis in the individual.

### Compositions

Aspects of the present disclosure further include compositions. In some embodiments, the compositions find use, e.g., in practicing the methods of the present disclosure.

In certain embodiments, a composition of the present disclosure comprises one or more drugs described elsewhere herein, including any of the drugs from Tables 2, 3 or 4. Non-limiting examples of such drugs which may be comprised in a composition (e.g., a pharmaceutical composition) include flumetasone, primaquine, flunisolide, zuclopenthixol, irinotecan, scopolamine, promazine, or any combination thereof.

According to some embodiments, a composition of the present disclosure includes the one or more drugs present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethy]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Pharmaceutical compositions are also provided. The pharmaceutical compositions of the present disclosure include one or more drugs from Tables 2, 3 or 4 and a pharmaceutically acceptable carrier. Any pharmaceutical composition of the present disclosure may include—in addition to the one or more drugs from Tables 2, 3 or 4—an additional agent that finds use, e.g., in treating endometriosis, e.g., a GnRH agonist (e.g., Leuprolide acetate (Lupron), Goserelin acetate (Zoladex), Nafarelin acetate (Synarel), and/or the like), a progestin (e.g., depot medroxyprogesterone acetate (Depo-Provera), norethindrone acetate (Aygestin), and/or the like), Danazol, or any combination thereof.

The one or more drugs from Tables 2, 3 or 4 can be incorporated into a variety of formulations for administration to an individual. More particularly, the one or more drugs from Tables 2, 3 or 4 can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the one or more drugs from Tables 2, 3 or 4 suitable for administration to an individual (e.g., suitable for human administration) are generally sterile and may further be free of detectable pyrogens or other contaminants contraindicated for administration to an individual according to a selected route of administration. The following methods and carriers/excipients are merely examples and are in no way limiting.

For oral preparations, the one or more drugs from Tables 2, 3 or 4 can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The one or more drugs from Tables 2, 3 or 4 can be formulated for parenteral (e.g., intravenous, intra-arterial, intraosseous, intramuscular, intracerebral, intracerebroventricular, intracranial, intrathecal, subcutaneous, etc.) administration. In some embodiments, the one or more drugs from Tables 2, 3 or 4 is formulated for intrauterine (e.g., in the form of an intrauterine device (IUD) comprising the one or more drugs releasable therefrom), intravaginal (e.g., in the form of a vaginal suppository or vaginal ring comprising the one or more drugs releasable therefrom), oral, parenteral, intranasal, intrathecal, or transdermal administration. In some embodiments, the one or more drugs from Tables 2, 3 or 4 is formulated for injection by dissolving, suspending or emulsifying the one or more drugs in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the one or more drugs from Tables 2, 3 or 4 may be prepared by mixing the one or more drugs having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, omithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the one or more drugs from Tables 2, 3 or 4 may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the one or more drugs from Tables 2, 3 or 4 against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the one or more drugs from Tables 2, 3 or 4, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

Kits

Also provided by the present disclosure are kits. In some embodiments, the kits find use, e.g., in practicing the methods of the present disclosure.

In some embodiments, a kit of the present disclosure includes one or more of any of the drugs described elsewhere herein, including one or more drugs from Tables 2, 3 or 4. Non-limiting examples of such drugs which may be included in a kit of the present disclosure include flumetasone, primaquine, flunisolide, zuclopenthixol, irinotecan, scopolamine, promazine, or any combination thereof.

In some embodiments, a kit of the present disclosure includes a pharmaceutical composition including one or more drugs from Tables 2, 3 or 4 and a pharmaceutically acceptable carrier. For example, provided are kits that include any of the pharmaceutical compositions of the present disclosure, including any of the pharmaceutical compositions described in the Compositions section hereinabove. In some embodiments, a kit of the present disclosure includes a pharmaceutical composition that—in addition to the one or more drugs from Tables 2, 3 or 4—further includes an additional agent that finds use, e.g., in treating endometriosis, e.g., a GnRH agonist (e.g., Leuprolide acetate (Lupron), Goserelin acetate (Zoladex), Nafarelin acetate (Synarel), and/or the like), a progestin (e.g., depot medroxyprogesterone acetate (Depo-Provera), norethindrone acetate (Aygestin), and/or the like), Danazol, or any combination thereof.

Kits for practicing the subject methods may include a quantity of the one or more drugs from Tables 2, 3 or 4 (and optionally, an additional agent as described above), present in unit dosages, e.g., IUDs, vaginal suppositories, vaginal rings, ampoules, tablets, capsules, or a multi-dosage format. As such, in certain embodiments, the kits may include one or more (e.g., two or more) unit dosages (e.g., IUDs, vaginal suppositories, vaginal rings, ampoules, tablets, capsules) of a pharmaceutical composition that includes the one or more drugs from Tables 2, 3 or 4.

The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular one or more drugs from Tables 2, 3 or 4 employed, the effect to be achieved, and the pharmacodynamics associated with the one or more drugs, in the individual. In yet other embodiments, the kits may include a single multi dosage amount of a composition including the one or more drugs from Tables 2, 3 or 4 (and optionally, an additional agent as described above).

Components of the kits may be present in separate containers, or multiple components may be present in a single container. For example, in a kit that includes two or more drugs from Tables 2, 3, or 4, the two or more drugs may be provided in the same composition (e.g., in one or more containers) or may be provided in separate compositions in separate containers. Suitable containers include individual tubes (e.g., vials), ampoules, sealed packages (e.g., containing one or more IUDs, vaginal suppositories, vaginal rings, ampoules, tablets, capsules, and/or the like), etc.

A kit of the present disclosure may further include instructions. For example, a kit that includes one or more drugs from Tables 2, 3 or 4 may include instructions for administering the one or more drugs (e.g., present in one or more pharmaceutical compositions) to an individual identified as having endometriosis.

The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:
1. A method of modulating gene expression levels in an individual identified as having endometriosis, the method comprising:
   administering to the individual identified as having endometriosis a drug from Table 2 in an amount effective to modulate gene expression levels in the individual.
2. The method according to embodiment 1, wherein two or more drugs from Table 2 are administered to the individual in an amount effective to modulate gene expression levels in the individual.
3. The method according to embodiment 1 or embodiment 2, wherein the drug is selected from the group consisting of: fenoprofen, flumetasone, flunisolide, zuclopenthixol, irinotecan, primaquine, scopolamine, promazine, sulfamethoxazole, and bepridil.
4. The method according to embodiment 1 or embodiment 2, wherein prior to the administering, the individual has been identified as having stage ½ endometriosis or stage ¾ endometriosis.
5. The method according to embodiment 4, wherein prior to the administering, the individual has been identified as having stage ½ endometriosis.
6. The method according to embodiment 5, comprising administering to the individual identified as having stage ½ endometriosis a drug from Table 3 in an amount effective to modulate gene expression levels in the individual.
7. The method according to embodiment 6, wherein the drug is selected from the group consisting of: fenoprofen, irinotecan, scopolamine, flumetasone, promazine, flunisolide, levonorgestrel, primaquine, ginkgolide A, and isoconazole.
8. The method according to embodiment 4, wherein prior to the administering, the individual has been identified as having stage ¾ endometriosis.
9. The method according to embodiment 8, comprising administering to the individual identified as having stage ¾ endometriosis a drug from Table 4 in an amount effective to modulate gene expression levels in the individual.
10. The method according to embodiment 9, wherein the drug is selected from the group consisting of: flumetasone, scopolamine, fenoprofen, irinotecan, zuclopenthixol, levonorgestrel, primaquine, flunisolide, promazine, and cloperastine.
11. The method according to any one of embodiments 1 to 10, wherein the administration is by oral administration.
12. The method according to any one of embodiments 1 to 10, wherein the administration is by parenteral administration.
13. The method according to embodiment 12, wherein the parenteral administration is by intrauterine administration.
14. The method according to embodiment 13, wherein the intrauterine administration comprises placement of an intrauterine device (IUD) comprising the drug in the uterus of the individual.
15. The method according to embodiment 12, wherein the parenteral administration is by intravaginal administration.
16. The method according to embodiment 15, wherein the intravaginal administration comprises administering a vaginal suppository comprising the drug to the individual.
17. The method according to embodiment 15, wherein the intravaginal administration comprises administering a vaginal ring comprising the drug to the individual.
18. The method according to any one of embodiments 1 to 17, wherein the modulation of gene expression results in at least an amelioration of one or more symptoms of endometriosis in the individual.
19. The method according to embodiment 18, wherein the one or more symptoms of endometriosis comprise dysmenorrhea, infertility, chronic pelvic pain, abnormal uterine bleeding or spotting, and any combination thereof.
20. The method according to any one of embodiments 1 to 19, further comprising identifying the individual as having endometriosis.
21. A pharmaceutical composition, comprising:
   a drug from Table 2 in an amount effective to modulate gene expression levels in an individual, wherein the pharmaceutical composition is adapted for intrauterine or intravaginal administration of the drug to the individual.
22. The pharmaceutical composition of embodiment 21, wherein the pharmaceutical composition is adapted for intrauterine administration of the drug to the individual.
23. The pharmaceutical composition of embodiment 22, wherein the composition is an intrauterine device (IUD).
24. The pharmaceutical composition of embodiment 21, wherein the pharmaceutical composition is adapted for intravaginal administration of the drug to the individual.
25. The pharmaceutical composition of embodiment 24, wherein the composition is a vaginal suppository.
26. The pharmaceutical composition of embodiment 24, wherein the composition is a vaginal ring.
27. The pharmaceutical composition of any one of embodiments 21 to 26, wherein the drug is selected from the group consisting of: fenoprofen, flumetasone, flunisolide, zuclopenthixol, irinotecan, primaquine, scopolamine, promazine, sulfamethoxazole, and bepridil.
28. The pharmaceutical composition of any one of embodiments 21 to 26, wherein the drug is selected from the group consisting of: fenoprofen, irinotecan, scopolamine, flumetasone, promazine, flunisolide, levonorgestrel, primaquine, ginkgolide A, and isoconazole.
29. The pharmaceutical composition of any one of embodiments 21 to 26, wherein the drug is selected from the group consisting of: flumetasone, scopolamine, fenoprofen, irinotecan, zuclopenthixol, levonorgestrel, primaquine, flunisolide, promazine, and cloperastine.
30. A kit comprising:
   a pharmaceutical composition comprising a drug from Table 2 in an amount effective to modulate gene expression levels in the individual; and
   instructions for administering the pharmaceutical composition to an individual identified as having endometriosis.
31. The kit of embodiment 30, comprising the pharmaceutical composition present in one or more unit dosages.
32. The kit of embodiment 30, comprising the pharmaceutical composition present in two or more unit dosages.
33. The kit of any one of embodiments 30 to 32, wherein the pharmaceutical composition is adapted for parenteral administration of the drug to the individual.
34. The kit of embodiment 33, wherein the pharmaceutical composition is adapted for intrauterine administration of the drug to the individual.
35. The kit of embodiment 34, wherein the pharmaceutical composition is an intrauterine device (IUD) comprising the drug.
36. The kit of embodiment 33, wherein the pharmaceutical composition is adapted for intravaginal administration of the drug to the individual.
37. The kit of embodiment 36, wherein the pharmaceutical composition is a vaginal suppository comprising the drug.
38. The kit of embodiment 36, wherein the pharmaceutical composition is a vaginal ring comprising the drug.
39. The kit of any one of embodiments 30 to 32, wherein the pharmaceutical composition is adapted for oral administration of the drug to the individual.
40. The kit of any one of embodiments 30 to 39, wherein the drug is selected from the group consisting of: fenoprofen, flumetasone, flunisolide, zuclopenthixol, irinotecan, primaquine, scopolamine, promazine, sulfamethoxazole, and bepridil.
41. The kit of any one of embodiments 30 to 39, wherein the drug is selected from the group consisting of: fenoprofen, irinotecan, scopolamine, flumetasone, promazine, flunisolide, levonorgestrel, primaquine, ginkgolide A, and isoconazole.
42. The kit of any one of embodiments 30 to 39, wherein the drug is selected from the group consisting of: flumetasone, scopolamine, fenoprofen, irinotecan, zuclopenthixol, levonorgestrel, primaquine, flunisolide, promazine, and cloperastine.

The following examples are offered by way of illustration and not by way of limitation.

### EXPERIMENTAL

### Example 1—Identification of Novel Therapeutic Candidates for Endometriosis

In this example, a transcriptomics based computational drug repurposing pipeline [3] was applied to endometriosis gene expression data in order to identify potential new therapeutics from existing drugs based on expression reversal. In addition, the study aimed to determine concordance of therapeutic predictions between disease stages and menstrual cycle phases to identify whether different subtypes of disease should be treated with different drugs or whether different drugs should be administered to endometriosis patients at different points of the cycle.

Methods

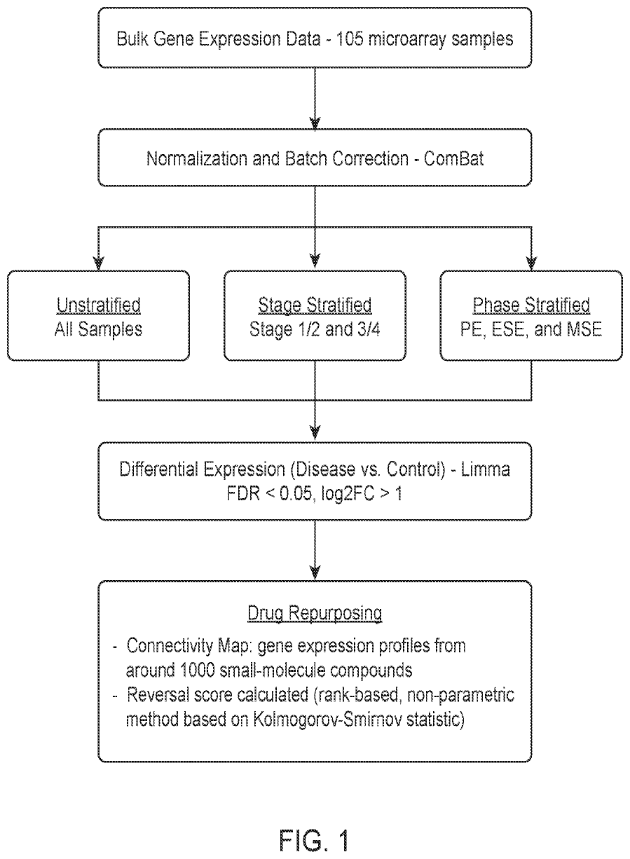

A flow diagram providing an overview of the pipeline employed in this example is shown in FIG. 1. Bulk gene expression data was obtained from 105 microarray samples from pristine control patients and patients with stage ½ and ¾ endometriosis at the early secretory (ESE), mid-secretory (MSE), and proliferative (PE) phases in the cycle. This data was normalized with the R package justRMA [4] and batch corrected using the package ComBat [5]. Using the package limma, [6] differential gene expression analysis between disease and control samples was performed on the unstratified data and the data stratified by cycle phase and disease stage. The significant genes from each signature were identified using the cutoffs adj P-val<0.05 and log FC>1.1 (Table 1).

A transcriptomics-based drug repurposing pipeline was then applied to the six signatures. The pipeline utilized a rank-based pattern-matching method, leveraging gene expression data for both diseases and drugs, in order to identify disease-drug pairs with opposite transcriptional effects. On the drug side, the Connectivity Map (CMAP) [7] dataset from the Broad Institute was used, which consists of gene expression profiles from over 1000 small-molecule drugs. Reversal scores were calculated for each drug in the CMAP dataset and permutation analysis was carried out to assess significance. Drug hits with q-values<0.0001 or reversal scores<0 (indicating signature reversal) were examined further resulting in 200-300 drug hits per signature (Table 1). Network analysis on DrugBank data was used to visualize the relationships between the top significant hits [8].

TABLE 1
Significant Genes and Total Drug Hits

| | Significant Genes | Total Drug Hits |
|---|---|---|
| | (adj P-val < 0.05, | (q-value < 0.0001, |
| | logFC > 1.1) | reversal score < 0) |
| Unstratified | 324 genes | 255 drug hits |
| Stage 1/2 | 571 genes | 270 drug hits |
| Stage 3/4 | 284 genes | 270 drug hits |
| PE | 645 genes | 264 drug hits |
| ESE | 171 genes | 190 drug hits |
| MSE | 456 genes | 291 drug hits |

Drugs having reversal scores (unstratified) of less than −0.40 are provided in Table 2 below.

TABLE 2
Unstratified Reversal (<−0.40)

| Drug Name | Unstratified | ATC Code |
|---|---|---|
| fenoprofen | −0.769908789 | M01AE04 |
| flumetasone | −0.767173301 | D07AB03 |
| flunisolide | −0.727467932 | R01AD04 |
| zuclopenthixol | −0.72613164 | N05AF05 |
| irinotecan | −0.724635538 | L01XX19 |
| primaquine | −0.719256369 | P01BA03 |
| scopolamine | −0.70554238 | N05CM05 |
| promazine | −0.69911679 | N05AA03 |
| sulfamethoxazole | −0.666700669 | J04AM08 |
| bepridil | −0.665567541 | C08EA02 |
| trifluoperazine | −0.64890725 | N05AB06 |
| levonorgestrel | −0.640962606 | G03AB03 |
| cloperastine | −0.636713165 | R05DB21 |
| medrysone | −0.63606287 | S01BA08 |
| camptothecin | −0.635593638 | |
| sertaconazole | −0.622460239 | G01AF19 |
| metolazone | −0.621820995 | C03EA12 |
| isoconazole | −0.621518374 | G01AF07 |
| procyclidine | −0.61342752 | N04AA04 |
| sulfadiazine | −0.594098046 | J01EE06 |
| fluorometholone | −0.587206624 | D07XB04 |
| cycloserine | −0.583742636 | J04AB01 |
| simvastatin | −0.583270854 | A10BH51 |
| pyrvinium | −0.580274399 | P02CX01 |
| ouabain | −0.579174423 | C01AC01 |
| valproic acid | −0.57650099 | N03AG01 |
| resveratrol | −0.575398465 | |
| nocodazole | −0.569991244 | |
| remoxipride | −0.566164282 | N05AL04 |
| rimexolone | −0.560672906 | H02AB12 |
| iopanoic acid | −0.560099117 | V08AC06 |
| ethotoin | −0.558729673 | N03AB01 |
| disopyramide | −0.551447224 | C01BA03 |
| zomepirac | −0.550587815 | M01AB04 |
| adipiodone | −0.547092376 | |
| cefazolin | −0.545064987 | J01DB04 |
| galantamine | −0.542792781 | N06DA04 |
| ginkgolide A | −0.534390806 | |
| cefuroxime | −0.534099236 | S01AA27 |
| estradiol | −0.532917655 | G03FA16 |
| demeclocycline | −0.529809842 | J01AA01 |
| fenbufen | −0.529432416 | M01AE05 |
| menadione | −0.525358087 | B02BA02 |
| felodipine | −0.519888812 | C07FB02 |
| clomipramine | −0.519172213 | N06AA04 |
| trazodone | −0.518381659 | N06AX05 |
| terfenadine | −0.51400811 | R06AX12 |
| niclosamide | −0.513318712 | P02DA01 |
| diethylcarbamazine | −0.51313425 | P02CB02 |
| metaraminol | −0.511494487 | C01CA09 |
| sirolimus | −0.511299824 | S01XA23 |
| ipratropium | −0.508342471 | |
| bromide | | |
| oxybuprocaine | −0.499900543 | S01HA02 |
| azacitidine | −0.496474809 | L01BC07 |
| hexestrol | −0.496156037 | |
| fluspirilene | −0.495978375 | N05AG01 |
| mebendazole | −0.495893369 | P02CA51 |
| fluphenazine | −0.495793912 | N05AB02 |
| procaine | −0.495646852 | N01BA52 |
| griseofulvin | −0.495262625 | D01BA01 |
| thalidomide | −0.49488945 | L04AX02 |
| latamoxef | −0.494348813 | J01DD06 |
| alsterpaullone | −0.494128648 | |
| acetylsalicylic acid | −0.489974413 | B01AC06 |
| methyldopa | −0.488532714 | |
| daunorubicin | −0.48788837 | L01DB02 |
| apomorphine | −0.48759 | G04BE07 |
| thiocolchicoside | −0.487355384 | M03BX05 |
| thioridazine | −0.486981358 | N05AC02 |
| pentolonium | −0.486444971 | |
| tobramycin | −0.485924736 | S01AA12 |
| hesperetin | −0.485681619 | |
| cyproheptadine | −0.484142164 | R06AX02 |
| pramocaine | −0.482908729 | D04AB07 |
| tretinoin | −0.482178529 | D10AD51 |
| bisacodyl | −0.481631942 | A06AB52 |
| methazolamide | −0.479689559 | G01AE10 |
| chlorprothixene | −0.479171023 | N05AF03 |
| chlorcyclizine | −0.479062216 | R06AE04 |
| piracetam | −0.478659288 | N06BX03 |
| trimipramine | −0.478455274 | N06AA06 |
| 8-azaguanine | −0.478368568 | |
| diltiazem | −0.476899668 | C08DB01 |
| saquinavir | −0.4766404 | J05AE01 |
| amoxicillin | −0.475843895 | A02BD04 |
| lomefloxacin | −0.475539574 | S01AE04 |
| haloperidol | −0.474533956 | N05AD01 |
| clioquinol | −0.471716013 | G01AC02 |
| fulvestrant | −0.471170275 | L02BA03 |
| azlocillin | −0.470736745 | J01CR50 |
| canrenoic acid | −0.46978383 | C03DA02 |
| methotrexate | −0.468263926 | L04AX03 |
| miconazole | −0.468100715 | A07AC01 |
| nifedipine | −0.467985957 | C08CA55 |
| digoxin | −0.466338544 | C01AA05 |
| ifenprodil | −0.465587943 | C04AX28 |
| natamycin | −0.46306242 | G01AA02 |
| rescinnamine | −0.462051701 | C02LA52 |
| flucytosine | −0.461399706 | J02AX01 |
| prenylamine | −0.460896471 | C01DX52 |
| oxybutynin | −0.460662705 | G04BD04 |
| sulconazole | −0.458842731 | D01AC09 |
| roxithromycin | −0.455757019 | J01FA06 |
| tolbutamide | −0.455521553 | V04CA01 |
| carbamazepine | −0.455221483 | N03AF01 |
| dipyridamole | −0.45460349 | B01AC07 |
| mesalazine | −0.454566088 | A07EC02 |
| astemizole | −0.453684577 | R06AX11 |
| fluocinonide | −0.453047884 | C05AA11 |
| ganciclovir | −0.450741676 | J05AB06 |
| sulfamerazine | −0.449288076 | J01ED07 |
| minaprine | −0.449219222 | N06AX07 |
| sulfadimidine | −0.448717687 | |
| pivampicillin | −0.446578091 | J01CR50 |
| trioxysalen | −0.445420311 | |
| etomidate | −0.442028579 | N01AX07 |
| chlorpropamide | −0.441435238 | A10BB02 |
| levamisole | −0.440945605 | P02CE01 |
| pyrazinamide | −0.440305511 | J04AK01 |
| mefenamic acid | −0.438619845 | M01AG01 |
| naproxen | −0.438517838 | G02CC02 |
| gliclazide | −0.438318075 | A10BB09 |
| aminocaproic acid | −0.436895927 | B02AA01 |
| pimozide | −0.436663011 | N05AG02 |
| naloxone | −0.436308537 | A06AH04 |
| xylometazoline | −0.436104523 | R01AB06 |
| labetalol | −0.435718597 | C07BG01 |
| prochlorperazine | −0.433375836 | N05AB04 |
| phenoxybenzamine | −0.433214325 | C04AX02 |
| corbadrine | −0.432203606 | |
| nortriptyline | −0.430981222 | N06AA10 |
| rolitetracycline | −0.429689134 | J01AA20 |
| monobenzone | −0.427521485 | D11AX13 |
| fluvoxamine | −0.426401108 | N06AB08 |
| proxymetacaine | −0.42628125 | |
| mitoxantrone | −0.425522998 | L01DB07 |
| sotalol | −0.425364887 | C07AA07 |
| tranylcypromine | −0.423516011 | N06AF04 |
| difenidol | −0.420680217 | |
| rosiglitazone | −0.419298872 | A10BD04 |
| hydralazine | −0.419071056 | C02DB02 |
| paracetamol | −0.418015284 | |
| mepyramine | −0.415635971 | D04AA02 |
| flufenamic acid | −0.413470873 | M01AG03 |
| omeprazole | −0.413010141 | A02BC01 |
| berberine | −0.412679469 | |
| phentolamine | −0.411800508 | V03AB36 |
| amiloride | −0.411548891 | C03DB01 |
| indometacin | −0.410349459 | |
| perhexiline | −0.410025587 | C08EX02 |
| cyanocobalamin | −0.409104974 | B03BA01 |
| pargyline | −0.40890691 | C02LL01 |
| cetirizine | −0.408548186 | R06AE07 |
| pioglitazone | −0.408484431 | A10BD09 |
| clomifene | −0.407223795 | G03GB02 |
| ritodrine | −0.406872721 | G02CA01 |
| bromopride | −0.405374918 | A03FA04 |
| clozapine | −0.405279712 | N05AH02 |
| aminophenazone | −0.404941388 | N02BB53 |
| carbachol | −0.40482238 | |
| vorinostat | −0.404514659 | L01XX38 |
| nalidixic acid | −0.403460587 | J01MB02 |
| paroxetine | −0.402513622 | N06AB05 |
| etidronic acid | −0.40248897 | M05BB01 |
| benzthiazide | −0.402369112 | G01AE10 |
| indapamide | −0.402002737 | C03BA11 |
| lymecycline | −0.401430648 | J01AA04 |
| amiodarone | −0.401192632 | C01BD01 |

The top 30 drug candidates by reversal scare for stage ½ and stage ¾ endometriosis are shown in Tables 3 and 4 below, respectively.

TABLE 3
Stage 1/2 (lowest 30 reversal scores)

| Drug Name | Stage 1/2 Reversal | ATC Code |
|---|---|---|
| fenoprofen | −0.855481393 | M01AE04 |
| irinotecan | −0.738573221 | L01XX19 |
| scopolamine | −0.731113847 | N05CM05 |
| flumetasone | −0.726532749 | D07AB03 |
| promazine | −0.705904105 | N05AA03 |
| flunisolide | −0.703026667 | R01AD04 |
| levonorgestrel | −0.697417015 | G03AB03 |
| primaquine | −0.695327717 | P01BA03 |
| ginkgolide A | −0.681040303 | |
| isoconazole | −0.676670382 | G01AF07 |
| zuclopenthixol | −0.64104325 | N05AF05 |
| procyclidine | −0.638042391 | N04AA04 |
| metolazone | −0.624333435 | C03EA12 |
| medrysone | −0.615506354 | S01BA08 |
| ethotoin | −0.610990652 | N03AB01 |
| cloperastine | −0.609434898 | R05DB21 |
| camptothecin | −0.603386779 | |
| galantamine | −0.600670804 | N06DA04 |
| sulfamethoxazole | −0.598542732 | J04AM08 |
| fluorometholone | −0.597667933 | D07XB04 |
| valproic acid | −0.593051972 | N03AG01 |
| nocodazole | −0.592821098 | |
| trifluoperazine | −0.59259558 | N05AB06 |
| cycloserine | −0.59098042 | J04AB01 |
| fenbufen | −0.588902803 | M01AE05 |
| demeclocycline | −0.588106938 | J01AA01 |
| sulfadiazine | −0.584348581 | J01EE06 |
| disopyramide | −0.58395315 | C01BA03 |
| sertaconazole | −0.579063886 | G01AF19 |
| remoxipride | −0.576128573 | N05AL04 |

TABLE 4
Stage 3/4 (lowest 30 reversal scores)

| Drug Name | Stage 3/4 Reversal | ATC Code |
|---|---|---|
| flumetasone | −0.818801 | D07AB03 |
| scopolamine | −0.7938726 | N05CM05 |
| fenoprofen | −0.7872555 | M01AE04 |
| irinotecan | −0.7642097 | L01XX19 |
| zuclopenthixol | −0.7563124 | N05AF05 |
| levonorgestrel | −0.7527998 | G03AB03 |
| primaquine | −0.7468475 | P01BA03 |
| flunisolide | −0.7259509 | R01AD04 |
| promazine | −0.7148608 | N05AA03 |
| cloperastine | −0.7049425 | R05DB21 |
| sulfamethoxazole | −0.6937 | J04AM08 |
| isoconazole | −0.6715971 | G01AF07 |
| medrysone | −0.6673933 | S01BA08 |
| sertaconazole | −0.6665638 | G01AF19 |
| galantamine | −0.6496283 | N06DA04 |
| demeclocycline | −0.6481662 | J01AA01 |
| bepridil | −0.6354649 | C08EA02 |
| simvastatin | −0.6313625 | A10BH51 |
| metolazone | −0.6298875 | C03EA12 |
| disopyramide | −0.6284883 | C01BA03 |
| sulfadiazine | −0.6265293 | J01EE06 |
| trifluoperazine | −0.620712 | N05AB06 |
| cycloserine | −0.6153748 | J04AB01 |
| thiocolchicoside | −0.6107986 | M03BX05 |
| remoxipride | −0.6027169 | N05AL04 |
| ginkgolide A | −0.6007179 | |
| valproic acid | −0.5991247 | N03AG01 |
| nocodazole | −0.5979405 | |
| procyclidine | −0.5973312 | N04AA04 |
| resveratrol | −0.592229011 | |

Results

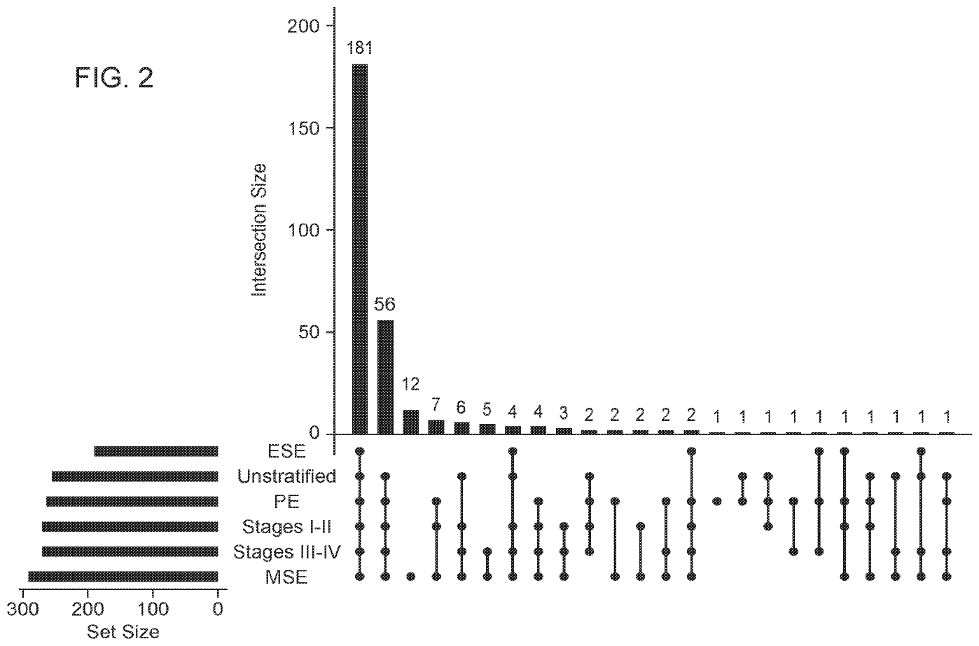

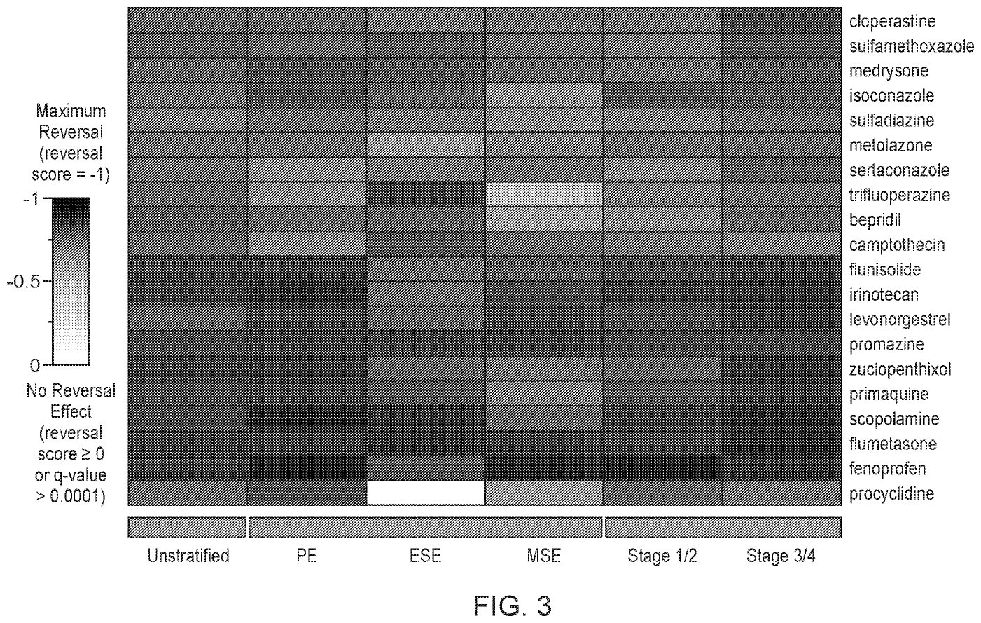

High levels of overlap in the drug hits for each signature were observed FIG. 2 despite the similarities not being as pronounced on the gene side. 181 of 298 total drug hits were common across all six signatures. There was an extensive amount of overlap between the top 20 drugs across all six signatures, with many drugs returning high reversal scores across the board (FIG. 3). Several of the identified drug hits have been used in clinical trials to treat endometriosis; three interesting examples include fenoprofen (average reversal score: −0.799), an NSAID commonly used for pain relief and used successfully to treat dysmenorrhea, a symptom of endometriosis [9]; levonorgestrel (average reversal score: −0.705), a progesterone-based emergency contraceptive also known as Plan B which has also been applied to endometriosis successfully in clinical trials [10]; and menadione (average reversal score: −0.562), which has been used to alleviate pain in dysmenorrhea patients [11].

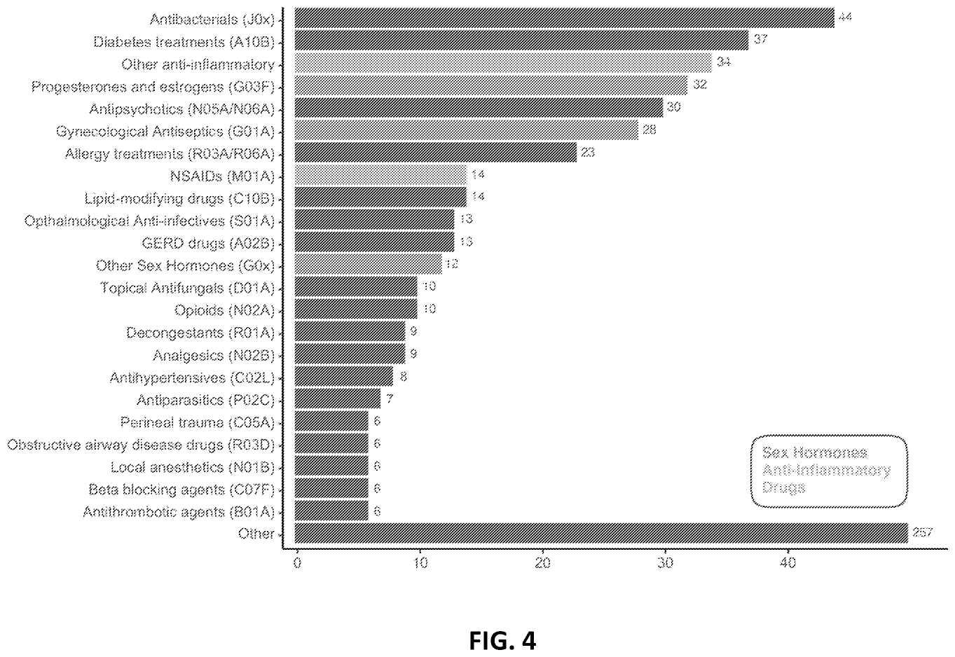

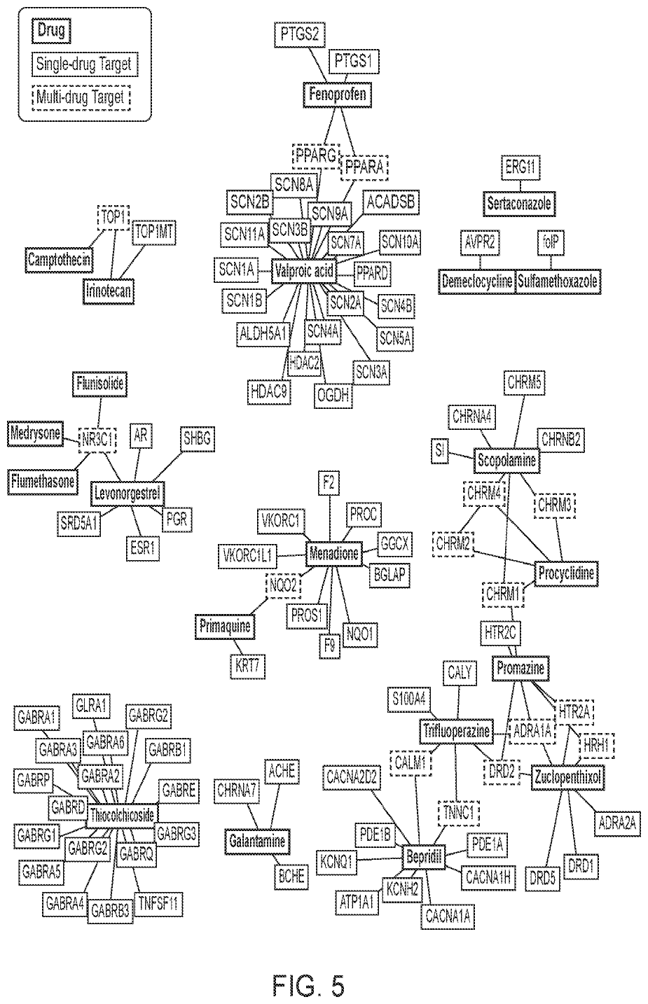

There were significant trends in the types of drugs returned by the pipeline, with two of the largest categories (anti-inflammatory and sex hormone drugs) being extensively used to treat endometriosis (FIG. 4). Upon looking into the protein interactions for the top 20 drugs (FIG. 5), 15 proteins were identified that were targeted by two or more drugs. Out of those proteins, three were found to have a direct link to endometriosis. PPARG and PPARA, which are commonly targeted by NSAIDS (including fenoprofen, which is shown in the network plot), inhibit the growth of endometrial tissue when activated [12] and NR3C1 is consistently expressed in endometrial cells [13]. Finally, leveraging this approach, a number of novel therapeutic candidates were identified such as flumetasone (average reversal score: −0.766), a corticosteroid meant for topical use [14], and primaquine (average reversal score: −0.697), an anti-malaria drug; a chemically similar drug, chloroquine, has been shown to have therapeutic effects on endometriosis [15].

In this work, a transcriptomics based drug repurposing approach was leveraged to identify known and novel therapeutic candidates for endometriosis. Therapeutic predictions were found to be consistent across disease stage and menstrual cycle phase and included many of the known treatments and novel candidates. The pipeline returned several drugs and drug categories with known therapeutic effects on endometriosis, lending credibility to the remaining results as novel therapeutics for endometriosis.

### Example 2—Efficacy of a Drug Identified by the Transcriptomics Based Computational Drug Repurposing Pipeline

Assessed in this example was the efficacy for treating endometriosis of an example drug identified by the transcriptomics based computational drug repurposing pipeline described in Example 1. In this particular example, the efficacy of Fenoprofen was assessed in a rat model of endometriosis. As detailed below, the data provide proof of concept that drugs identified in Example 1 as having an opposite transcriptional effect with respect to the endometriosis gene expression signature are reasonably expected to be efficacious in ameliorating one or more symptoms of endometriosis.

Baseline vaginal nociception was first assessed in rats. Then, endometriosis was induced when rats were four months of age by suturing uterine fragments within the peritoneal cavity to generate ectopic endometrium (lesions). Rats were surgically induced with endometriosis based on the model by Vernon and Wilson (1985) in the stage of diestrus. In this model, four (2 mm×2 mm) pieces of uterine horn are auto transplanted onto cascading mesenteric arteries within the pelvic cavity of rats that are hormonally and immunologically intact. The present model avoids ovariectomy followed by estradiol supplementation that is common in most mouse models which eliminates the natural estradiol variations within the estrous cycle and the ability to assess drug treatment effects on fertility. It has been shown before that endometriosis but not a sham-surgery (auto-transplantation of fat) induces vaginal hyperalgesia. In prior experience with this endometriosis model, 100% of the animals showed evidence of peritoneal endometriosis and developed vaginal hyperalgesia. The experiments compare treatment protocols, starting ten weeks after induction of endometriosis when lesion growth and vaginal hyperalgesia in this model are already stabilized. After four weeks of treatment, a terminal laparotomy is performed and lesions and peritoneal fluid collected in the proestrus phase of the estrous cycle, to ensure similar endocrine conditions. Fenoprofen treatment (30 mg/kg/day, p.o.), Ibuprofen treatment as a positive control (30 mg/kg, day, p.o.), as well as two negative control experiments (no surgery no treatment or surgery and no treatment) were carried out with six animals per group.

Behavioral assessment of vaginal nociception: Volumes of water (0.01, 0.15, 0.30, 0.40, 0.55, 0.70, 0.80, and 0.90 mL) were delivered via a vaginal balloon to the rat's vaginal canal. When the previously trained rat detects this stimulus as noxious, she will poke her head into a tube to break an infrared (IR) beam. This beam break terminates the stimulus (balloon deflates). During each testing session, eight different distention volumes were delivered randomly three times each ˜60 seconds apart. The maximum latency of 15 seconds is considered to be no response. The experimenter was blinded to the volumes being delivered.

Data analyses of behavioral results: Percent escape responses as a function of distention volume were measured in each session. For each rat, the escape response percentages as a function of distention volume from all sessions for each testing period were determined. The median escape response percentage and interquartile range (IQR) for all rats from each testing period was determined.

Differences in the escape responses between baseline and the post endo testing period, between post-endo period and post-treatment period, and between baseline and the post-treatment period were then calculated for the different testing conditions: (a) treatment with Fenoprofen, (b) treatment with Ibuprofen (positive control), (c) no surgery and no treatment, and (d) no treatment. Statistical analyses were performed using Mann Whitney U test, and p-values were corrected using the Bonferroni correction method. Behavioral data is reported in the estrous stage of proestrus when estradiol levels and endometriosis-induced vaginal hyperalgesia are greatest.

Results

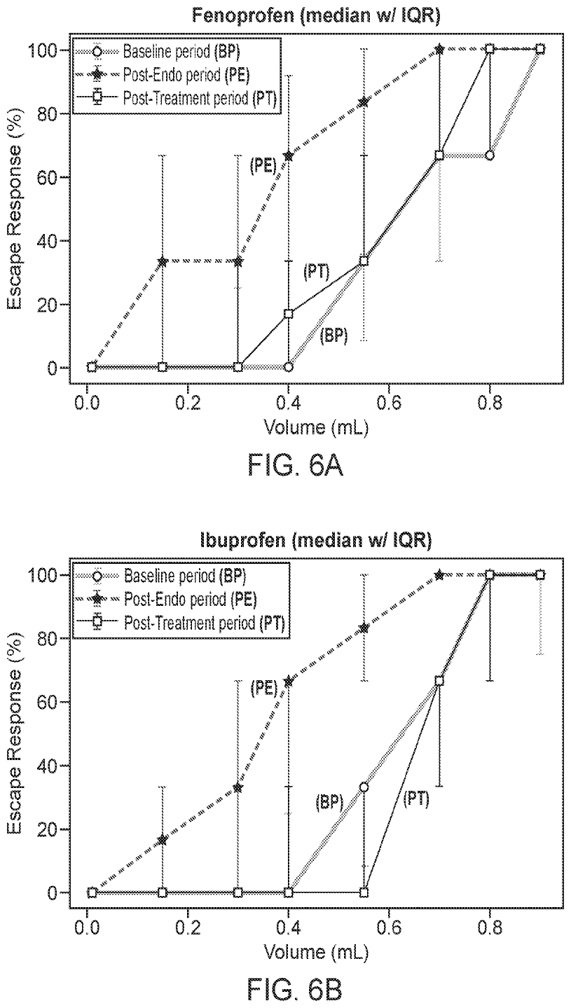

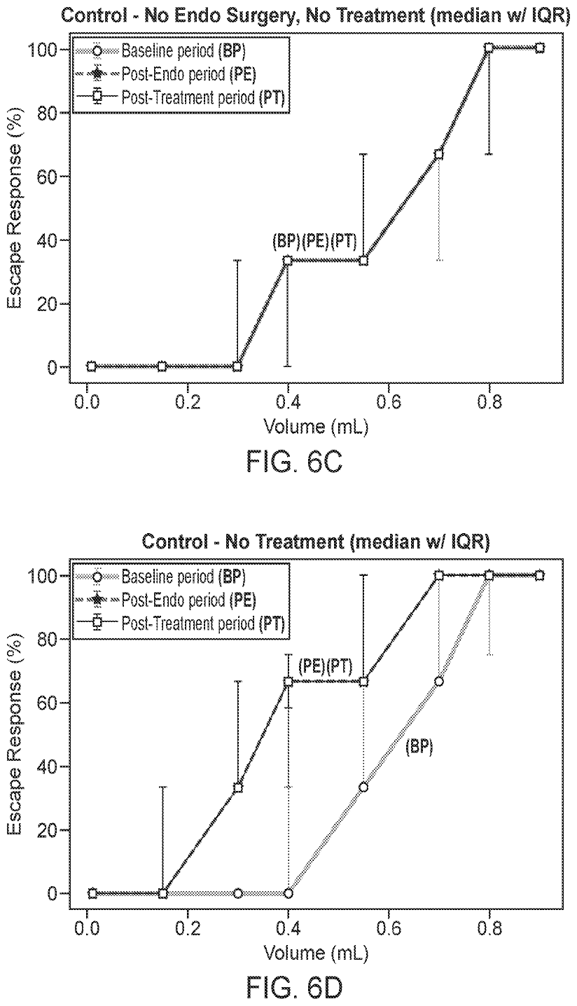

In an analysis of differences between escape responses among Fenoprofen (30 mg/kg/day, p.o.) treated subjects, escape responses were significantly increased during the post-endo surgery period compared to the baseline period, when volumes of 0.15, 0.3, 0.4, 0.55, 0.7, and 0.8 mL of water were delivered (Mann Whitney U test, Bonferroni-corrected p-value threshold of 0.05. FIG. 6A, Table 5). During the post-treatment period, escape responses were significantly decreased compared to the post-endo surgery period when volumes of 0.15, 0.3, 0.4, 0.55, 0.7, and 0.8 mL of water were delivered (Mann Whitney U test, Bonferroni-corrected p-value threshold of 0.05. FIG. 6A, Table 5). No statistically significant difference was found in the escape responses between the baseline period and the post-treatment period for any volume of water delivered to the Fenoprofen treated subjects (Mann Whitney U test, Bonferroni-corrected p-value threshold of 0.05. FIG. 6A, Table 5).

TABLE 5
Responses with Fenoprofen treatment. Median escape response with interquartile range for each delivered volume
(0.01, 0.15, 0.30, 0.40, 0.55, 0.70, 0.80, and 0.90 mL) during the baseline, post-endo surgery, and post-treatment
periods, with Bonferroni-corrected p-values from Mann-Whitney U test for baseline period vs post-endo surgery
period, post-endo surgery vs post-treatment period, and baseline period vs post-treatment period.

| | | | | | | | Baseline | Post-Endo | Baseline |
|---|---|---|---|---|---|---|---|---|---|
| | | | | | | | period vs Post- | period vs Post- | period vs Post- |
| | | | | | | | Endo | Treatment | Treatment |
| | | | Post- | | Post- | | period | period | period |
| | Baseline | | Endo | | Treatment | | MWU test | MWU test | MWU test |
| | period | | period | | period | | Bonferroni- | Bonferroni- | Bonferroni- |
| | (BL) | | (PE) | | (PT) | | adjusted | adjusted | adjusted |
| Volume | median | BL IQR | median | PE IQR | median | PT IQR | p-values | p-values | p-values |
| 0.01 | 0 | (0-0) | 0 | (0-0) | 0 | (0-0) | 1 | 1 | 1 |
| 0.15 | 0 | (0-0) | 33.3 | (0-66.6) | 0 | (0-0) | 2.7E−04* | 2.3E−03* | 0.61 |
| 0.3 | 0 | (0-25) | 33.3 | (33.3-66.6) | 0 | (0-33.3) | 2.0E−05* | 3.0E−06* | 1 |
| 0.4 | 0 | (0-33.3) | 66.6 | (33.3-91.7) | 16.65 | (0-33.3) | 4.0E−06* | 1.8E−05* | 1 |
| 0.55 | 33.3 | (8.3-33.3) | 83.3 | (66.6-100) | 33.3 | (33.3-66.6) | 2.7E−07* | 7.9E−07* | 0.16 |
| 0.7 | 66.6 | (33.3-66.6) | 100 | (100-100) | 66.6 | (66.6-100) | 1.5E−03* | 4.4E−02* | 0.97 |
| 0.8 | 66.6 | (66.6-100) | 100 | (100-100) | 100 | (66.6-100) | 4.8E−05* | 5.8E−03* | 1 |
| 0.9 | 100 | (100-100) | 100 | (100-100) | 100 | (100-100) | 0.34 | 0.80 | 1 |

*denotes Bonferroni-corrected p-values below significance threshold of 0.05.

Similarly, among Ibuprofen (30 mg/kg, day, p.o.) treated subjects (positive control), escape responses were significantly increased during the post-endo surgery period compared to the baseline period, when volumes of 0.15, 0.3, 0.4, 0.55, 0.7, and 0.8 mL of water were delivered (Mann Whitney U test, Bonferroni-corrected p-value threshold of 0.05. FIG. 6B, Table 6). During the post-treatment period, escape responses were significantly decreased compared to the post-endo surgery period, when volumes of 0.15, 0.3, 0.4, 0.55, 0.7, and 0.8 mL of water were delivered (Mann Whitney U test, Bonferroni-corrected p-value threshold of 0.05. FIG. 6B, Table 6). No statistically significant difference was found in the escape responses between the baseline period and the post-treatment period for any volume of water delivered to the Ibuprofen treated subjects (Mann Whitney U test, Bonferroni-corrected p-value threshold of 0.05. FIG. 6B, Table 6).

TABLE 6
Responses with Ibuprofen treatment (positive control). Median escape response with interquartile range for each delivered
volume (0.01, 0.15, 0.30, 0.40, 0.55, 0.70, 0.80, and 0.90 mL) during the baseline, post-endo surgery, and post-treatment
periods, with Bonferroni-corrected p-values from Mann-Whitney U test for baseline period vs post-endo surgery period,
post-endo surgery period vs post-treatment period, and baseline period vs post-treatment period.

| | | | | | | | Baseline | Post-Endo | Baseline |
|---|---|---|---|---|---|---|---|---|---|
| | | | | | | | period vs Post- | period vs Post- | period vs Post- |
| | | | | | | | Endo | Treatment | Treatment |
| | | | Post- | | Post- | | period | period | period |
| | Baseline | | Endo | | Treatment | | MWU period | MWU test | MWU test |
| | period | | period | | period | | Bonferroni- | Bonferroni- | Bonferroni- |
| | (BL) | | (PE) | | (PT) | | adjusted | adjusted | adjusted |
| Volume | median | BL IQR | median | PE IQR | median | PT IQR | p-values | p-values | p-values |
| 0.01 | 0 | (0-0) | 0 | (0-0) | 0 | (0-0) | 1 | 1 | 1 |
| 0.15 | 0 | (0-0) | 16.65 | (0-33.3) | 0 | (0-0) | 3.5E−03 * | 2.3E−03 * | 1 |
| 0.3 | 0 | (0-0) | 33.3 | (0-66.6) | 0 | (0-0) | 2.0E−03 * | 9.7E−04 * | 1 |
| 0.4 | 0 | (0-25) | 66.6 | (33.3-66.6) | 0 | (0-33.3) | 1.5E−05 * | 2.9E−06 * | 1 |
| 0.55 | 33.3 | (8.3-33.3) | 83.3 | (66.6-100) | 0 | (0-33.3) | 2.5E−07 * | 3.4E−08 * | 1 |
| 0.7 | 66.6 | (66.6-66.6) | 100 | (100-100) | 66.6 | (33.3-66.6) | 5.8E−05 * | 5.1E−06 * | 1 |
| 0.8 | 100 | (66.6-100) | 100 | (100-100) | 100 | (66.6-100) | 3.9E−03 * | 1.8E−03 * | 1 |
| 0.9 | 100 | (74.9-100) | 100 | (100-100) | 100 | (100-100) | 0.12 | 0.80 | 1 |

* denotes Bonferroni-corrected p-values below significance threshold of 0.05.

Among subjects that received neither endo surgery nor treatment, no statistically significant difference was found in the escape responses between the baseline and post-endo surgery periods, the post-endo surgery and post-treatment periods, or the baseline and post-treatment periods when any volume of water was delivered (Mann Whitney U test, Bonferroni-corrected p-value threshold of 0.05. FIG. 6C, Table 7).

TABLE 7
Responses with no endo surgery and no treatment (negative control). Median escape response with interquartile range
for each delivered volume (0.01, 0.15, 0.30, 0.40, 0.55, 0.70, 0.80, and 0.90 mL) during the baseline, post-endo surgery,
and post-treatment periods, with Bonferroni-corrected p-values from Mann-Whitney U test for baseline period vs post-
endo surgery period, post-endo surgery vs post-treatment period, and baseline period vs post-treatment period.

| | | | | | | | Baseline | Post-Endo | Baseline |
|---|---|---|---|---|---|---|---|---|---|
| | | | | | | | period vs Post- | period vs Post- | period vs Post- |
| | | | | | | | Endo | Treatment | Treatment |
| | | | Post- | | Post- | | period | period | period |
| | Baseline | | Endo | | Treatment | | MWU test | MWU test | MWU test |
| | period | | period | | period | | Bonferroni- | Bonferroni- | Bonferroni- |
| | (BL) | | (PE) | | (PT) | | adjusted | adjusted | adjusted |
| Volume | median | BL IQR | median | PE IQR | median | PT IQR | p-values | p-values | p-values |
| 0.01 | 0 | (0-0) | 0 | (0-0) | 0 | (0-0) | 1 | 1 | 1 |
| 0.15 | 0 | (0-0) | 0 | (0-0) | 0 | (0-0) | 1 | 1 | 1 |
| 0.3 | 0 | (0-0) | 0 | (0-33.3) | 0 | (0-33.3) | 1 | 1 | 1 |
| 0.4 | 33.3 | (0-33.3) | 33.3 | (0-33.3) | 33.3 | (0-33.3) | 1 | 1 | 1 |
| 0.55 | 33.3 | (33.3-33.3) | 33.3 | (33.3-66.6) | 33.3 | (33.3-66.6) | 1 | 1 | 1 |
| 0.7 | 66.6 | (33.3-66.6) | 66.6 | (66.6-66.6) | 66.6 | (66.6-66.6) | 1 | 1 | 1 |
| 0.8 | 100 | (66.6-100) | 100 | (66.6-100) | 100 | (66.6-100) | 1 | 1 | 1 |
| 0.9 | 100 | (100-100) | 100 | (100-100) | 100 | (100-100) | 1 | 1 | 1 |

* denotes Bonferroni-corrected p-values below significance threshold of 0.05.

Among subjects that received endo surgery but no treatment, escape responses were significantly increased during the post-endo surgery period compared to the baseline period, when volumes of 0.15, 0.3, 0.4, 0.55, and 0.7 mL of water were delivered (Mann Whitney U test, Bonferroni-corrected p-value threshold of 0.05. FIG. 6D, Table 8). During the post-treatment period, escape responses were also significantly increased compared to the baseline period when volumes of 0.3, 0.4, and 0.55 mL of water were delivered (Mann Whitney U test, Bonferroni-corrected p-value threshold of 0.05. FIG. 6D, Table 8). No statistically significant difference was found in the escape responses between the post-endo surgery period and the post-treatment period for any volume of water delivered to these subjects (Mann Whitney U test, Bonferroni-corrected p-value threshold of 0.05. FIG. 6D, Table 8).

TABLE 8
Responses with no treatment (positive control). Median escape response with interquartile range for each delivered
volume (0.01, 0.15, 0.30, 0.40, 0.55, 0.70, 0.80, and 0.90 mL) during the baseline, post-endo surgery, and post-
treatment periods, with Bonferroni-corrected p-values from Mann-Whitney U test for baseline period vs post-endo
surgery period, post-endo surgery period vs post-treatment period, and baseline period vs post-treatment period.

| | | | | | | | Baseline | Post-Endo | Baseline |
|---|---|---|---|---|---|---|---|---|---|
| | | | | | | | period vs Post- | period vs Post- | period vs Post- |
| | | | | | | | Endo | Treatment | Treatment |
| | | | Post- | | Post- | | period | period | period |
| | Baseline | | Endo | | Treatment | | MWU period | MWU period | MWU period |
| | period | | period | | period | | Bonferroni- | Bonferroni- | Bonferroni- |
| | (BL) | | (PE) | | (PT) | | adjusted | adjusted | adjusted |
| Volume | median | BL IQR | median | PE IQR | median | PT IQR | p-values | p-values | p-values |
| 0.01 | 0 | (0-0) | 0 | (0-0) | 0 | (0-0) | 1 | 1 | 1 |
| 0.15 | 0 | (0-0) | 0 | (0-33.3) | 0 | (0-33.3) | 0.02 * | 1 | 0.06 |
| 0.3 | 0 | (0-0) | 33.3 | (33.3-66.6) | 33.33 | (33.3-66.6) | 4E−05 * | 1 | 2E−06 * |
| 0.4 | 0 | (0-33.3) | 66.6 | (33.3-66.7) | 66.6 | (58.3-75) | 4E−06 * | 1 | 2E−05 * |
| 0.55 | 33.3 | (33.3-66.6) | 66.66 | (66.6-100) | 66.63 | (66.6-100) | 6E−06 * | 1 | 2E−05 * |
| 0.7 | 66.6 | (66.6-100) | 100 | (100-100) | 100 | (100-100) | 7E−03 * | 1 | 0.07 |
| 0.8 | 100 | (75-100) | 100 | (100-100) | 100 | (100-100) | 0.43 | 1 | 0.60 |
| 0.9 | 100 | (100-100) | 100 | (100-100) | 100 | (100-100) | 1 | 1 | 1 |

* denotes Bonferroni-corrected p-values below significance threshold of 0.05.

### REFERENCES

[1] Acog.org. 2020. Endometriosis. [online] Available at: <www.acog.org/patient-resources/faqs/gynecologic-problems/endometriosis>[Accessed 27 Aug. 2020].
[2] Wouters O J, McKee M, Luyten J. Estimated Research and Development Investment Needed to Bring a New Medicine to Market, 2009-2018. JAMA. 2020; 323(9):844-853. doi:10.1001/jama.2020.1166
[3] Sirota M, Dudley J T, Kim J, et al. Discovery and preclinical validation of drug indications using compendia of public gene expression data [published correction appears in Sci Transl Med. 2011 Sep. 28; 3(102):102er7]. Sci Transl Med. 2011; 3(96):96ra77. doi:10.1126/scitranslmed.3001318
[4] Gautier, L., Cope, L., Bolstad, B. M., and Irizarry, R. A. 2004. affy—analysis of Affymetrix GeneChip data at the probe level. Bioinformatics 20, 3 (February 2004), 307-315.
[5] Jeffrey T. Leek, W. Evan Johnson, Hilary S. Parker, Elana J. Fertig, Andrew E. Jaffe, Yuqing Zhang, John D. Storey and Leonardo Collado Torres (2020). sva: Surrogate Variable Analysis. R package version 3.36.0.
[6] Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Research 43(7), e47.
[7] J. Lamb et al., “The Connectivity Map: using gene-expression signatures to connect small molecules, genes, and disease,” Science, vol. 313, no. 5795, pp. 1929-1935, September 2006, doi: 10.1126/science.1132939.
[8] D. S. Wishart et al., “DrugBank 5.0: a major update to the DrugBank database for 2018,” Nucleic Acids Res., vol. 46, no. D1, pp. D1074-D1082, January 2018, doi: 10.1093/nar/gkx1037.
[9] National Center for Biotechnology Information. PubChem Compound Summary for CID 3342, Fenoprofen. pubchem.ncbi.nlm.nih.gov/compound/Fenoprofen. Accessed Aug. 27, 2020.
[10] National Center for Biotechnology Information. PubChem Compound Summary for CID 13109, Levonorgestrel. pubchem.ncbi.nlm.nih.gov/compound/Levonorgestrel. Accessed Aug. 27, 2020.
[11] National Center for Biotechnology Information. PubChem Compound Summary for CID 13109, Levonorgestrel. pubchem.ncbi.nlm.nih.gov/compound/Levonorgestrel. Accessed Aug. 27, 2020.
[12] Lebovic D I, Kavoussi S K, Lee J, Banu S K, Arosh J A. PPARγ activation inhibits growth and survival of human endometriotic cells by suppressing estrogen biosynthesis and PGE2 signaling. Endocrinology. 2013; 154(12):4803-4813. doi:10.1210/en.2013-1168
[13] Simmons R M, Satterfield M C, Welsh T H Jr, Bazer F W, Spencer T E. HSD11B1, HSD11B2, PTGS2, and NR3C1 expression in the peri-implantation ovine uterus: effects of pregnancy, progesterone, and interferon tau. Biol Reprod. 2010; 82(1):35-43. doi:10.1095/biolreprod.109.079608
[14] National Center for Biotechnology Information. PubChem Compound Summary for CID 16490, Flumethasone. pubchem.ncbi.nlm.nih.gov/compound/Flumethasone. Accessed Aug. 27, 2020.
[15] National Center for Biotechnology Information. PubChem Compound Summary for CID 4908, Primaquine. pubchem.ncbi.nlm.nih.gov/compound/Primaquine. Accessed Aug. 27, 2020.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of modulating gene expression levels in an individual identified as having endometriosis, the method comprising:
administering to the individual identified as having endometriosis a drug from Table 2 in an amount effective to modulate gene expression levels in the individual.

2. The method according to claim 1, wherein two or more drugs from Table 2 are administered to the individual in an amount effective to modulate gene expression levels in the individual.

3. The method according to claim 1 or claim 2, wherein prior to the administering, the individual has been identified as having stage ½ endometriosis or stage ¾ endometriosis.

4. The method according to claim 3, wherein prior to the administering, the individual has been identified as having stage ½ endometriosis.

5. The method according to claim 4, comprising administering to the individual identified as having stage ½ endometriosis a drug from Table 3 in an amount effective to modulate gene expression levels in the individual.

6. The method according to claim 3, wherein prior to the administering, the individual has been identified as having stage ¾ endometriosis.

7. The method according to claim 6, comprising administering to the individual identified as having stage ¾ endometriosis a drug from Table 4 in an amount effective to modulate gene expression levels in the individual.

8. The method according to any one of claims 1 to 7, wherein the parenteral administration is by intrauterine administration.

9. The method according to claim 8, wherein the intrauterine administration comprises placement of an intrauterine device (IUD) comprising the drug in the uterus of the individual.

10. The method according to any one of claims 1 to 7, wherein the parenteral administration is by intravaginal administration.

11. The method according to claim 10, wherein the intravaginal administration comprises administering a vaginal suppository comprising the drug to the individual.

12. The method according to claim 10, wherein the intravaginal administration comprises administering a vaginal ring comprising the drug to the individual.

13. The method according to any one of claims 1 to 12, wherein the modulation of gene expression results in at least an amelioration of one or more symptoms of endometriosis in the individual.

14. A pharmaceutical composition, comprising:
a drug from Table 2 in an amount effective to modulate gene expression levels in an individual, wherein the pharmaceutical composition is adapted for intrauterine or intravaginal administration of the drug to the individual.

15. The pharmaceutical composition of claim 14, wherein the pharmaceutical composition is adapted for intrauterine administration of the drug to the individual.

16. The pharmaceutical composition of claim 15, wherein the composition is an intrauterine device (IUD).

17. The pharmaceutical composition of claim 14, wherein the pharmaceutical composition is adapted for intravaginal administration of the drug to the individual.

18. The pharmaceutical composition of claim 17, wherein the composition is a vaginal suppository.

19. The pharmaceutical composition of claim 17, wherein the composition is a vaginal ring.

20. A kit comprising:
a pharmaceutical composition comprising a drug from Table 2 in an amount effective to modulate gene expression levels in the individual; and
instructions for administering the pharmaceutical composition to an individual identified as having endometriosis.
